(19) United States
(12) Patent Application Publication
(10) Pub. No.: US 2004/0185108 A1
(43) Pub. Date: Sep. 23, 2004

(54) Method of preparing gas-filled polymer matrix microparticles useful for delivering drug

(76) Inventors: Robert E. Short, Los Gatos, CA (US); Thomas B. Ottoboni, Belmont, CA (US)

Correspondence Address: POINT BIOMEDICAL CORPORATION, 887L Industrial Road, San Carlos, CA, 94070, US

(21) Appl. No.: 10/390,974

(22) Filed: Mar. 18, 2003

(51) Int. Cl. A61K 9/14

(52) U.S. Cl. 424/489

(57) ABSTRACT

A method is provided to prepare drug containing gas-filled porous microparticles having a polymer matrix interior which are useful for ultrasound mediated targeted delivery of a drug. An oil-in-water suspension is formed, both phases are frozen, then the aqueous and nonaqueous frozen phases are removed by sublimation.

### TECHNICAL FIELD

This invention pertains to drug delivering compositions and a method of preparing gas-filled microparticles having a polymer matrix interior which are useful for ultrasound mediated targeted delivery of a drug.

### BACKGROUND

Ultrasound is a modern medical imaging modality using sound energy to noninvasively visualize the interior structures and organs of a patient. Pulses of high frequency sound, generally in the megaHertz (MHz) range, emitted from a hand-held transducer are propagated into the body where they encounter different surfaces and interfaces. A portion of the incident sound energy is reflected back to the transducer that converts the sound waves into electronic signals which are then presented as a two-dimensional echographic image on a display monitor.

One of the advances in ultrasound imaging has been the development of ultrasonic contrast agents. Use of contrast agents enables the sonographer to visualize the vascular system which is otherwise relatively difficult to image. In cardiology for example, ultrasound contrast injected into the bloodstream permits the cardiologist to better visualize heart wall motion with the opacification of the heart chambers. Perhaps more importantly, contrast can be used to assess perfusion of blood into the myocardium to determine the location and extent of damage caused by an infarct. Similarly, visualization of blood flow using ultrasound contrast in other organs such as the liver and kidneys has found utility in diagnosing disease states in these organs.

Depending on the mechanical properties of the encapsulating material, microbubbles can be designed to rupture when exposed to ultrasound. Accordingly, a gas-filled microparticle that is rupturable when exposed to ultrasound also has potential in applications where site-specific delivery of a drug is desired.

The use of gas-filled ultrasound contrast agents serving also as drug carriers has been described for gas-filled liposomes in U.S. Pat. No. 5,580,575. A quantity of liposomes containing drug is administered into the circulatory system of a patient and monitored using ultrasonic energy at diagnostic levels until the presence of the liposomes is detected in the region of interest. Ultrasonic energy is then applied to the region at a power level that is sufficient to rupture the liposomes thus releasing the drug. The ultrasonic energy is described in U.S. Pat. No. 5,558,082 to be applied by a transducer that simultaneously applies diagnostic and therapeutic ultrasonic waves from transducer elements located centrally to the diagnostic transducer elements.

The use of gas-filled microcapsules to control the delivery of drugs to a region of the body has also been described in U.S. Pat. No. 5,190,766 in which the acoustic resonance frequency of the drug carrier is measured in the region in which the drug is to be released and then the region is irradiated with the appropriate sound wave to control the release of drug. Separate ultrasound transducers are described for the imaging and triggering of drug release in the target region.

### SUMMARY

This invention pertains to a novel drug containing gas-filled polymer matrix microparticles suitable for use as an ultrasound contrast agent and for the ultrasound mediated delivery of a drug and methods of preparation of same. A method of preparation comprises the steps of:

1. dissolving a polymer and a drug in a substantially water-immiscible solvent;

2. emulsifying the polymer/drug solution in an aqueous medium, optionally containing suitable surfactants, viscosity enhancers, and bulking agents;

3. reducing the temperature of the emulsion wherein both aqueous and water-immiscible phases become frozen;

4. removing water from the aqueous phase and solvent from water-immiscible phase by means of sublimation, resulting in the formation of drug containing polymer matrix microparticles;

5. introducing a gas into the polymer matrix microparticles.

Step 2 may be modified such that the aqueous medium also contains a biologically compatible amphiphilic material which encapsulates the emulsion droplets. Upon crosslinking, the amphiphilic material becomes a contiguous outer layer of the microparticle.

The method may also include, after step 2, the step of replacing the aqueous medium with a second aqueous medium. This additional step is useful when the components of an aqueous medium optimized for emulsion of the polymer solution are different from the components of an aqueous medium optimized for lyophilization. The additional step may be achieved by centrifugation or by diafiltration.

Also provided is a method of delivering a drug to an organ or tissue of a patient comprising the steps of:

1. injecting into a patient a suspension of drug-containing gas-filled polymer matrix microparticles in a physiologically acceptable aqueous liquid carrier where the microparticles have a mean size of about 1 to 10 microns and are made of a biodegradable synthetic polymer, and

2. applying an ultrasound signal to the organ or tissue at a power intensity sufficient to induce rupture and flooding of the microparticles, and

3. maintaining said power intensity until at least a substantial number of the microparticles are ruptured.

### DESCRIPTION OF DRAWINGS

In the accompanying drawings:

FIG. 1 is a plot of frame number vs. acoustic densitometry backscatter taken on an ultrasonic scanner as described in Example 7 for a test of the microparticles made in accordance with Example 1.

FIG. 2 is a plot of sound intensity in MI² vs. peak backscatter as described in Example 7.

FIG. 3 is a plot of sound intensity in MI² vs. fragility slope as described in Example 7.

### DETAILED DESCRIPTION

The present invention provides drug-containing gas-filled porous microparticles having a polymer matrix interior. Such microparticles are useful as an ultrasonic contrast agent and for site-specific delivery of a drug. These microparticles, being porous, rely on the hydrophobicity of the polymer to retain the gas within. The microparticles may be produced to also include an outer layer of a biologically compatible amphiphilic material, thus providing a surface for chemical modification to serve various purposes.

Microparticles can be fabricated to encapsulate both a drug and a gas. These microparticles can then be dispersed within the bloodstream and insonated with ultrasound at an intensity sufficient to cause the microparticles to rupture thereby releasing the drug into the surrounding medium. Thus, the circulating microparticles do not release their drug payload until they are triggered to do so using ultrasound. For example, a drug may be selectively delivered to heart tissue by first injecting intravenously a suspension of drug-containing microbubbles and then focusing an ultrasound beam on the heart to rupture the microbubbles that are perfusing the heart tissues. This type of drug delivery system is particularly advantageous when toxicity from systemic delivery of the drug is a concern. By limiting release of a pharmaceutical agent to a specific targeted site, toxic side effects can be minimized. In addition, total required dosage will typically be lower and result in a decrease in costs for the patient.

A class of therapeutic moieties deliverable by microbubbles triggered by ultrasound is chemotherapeutic agents used for the treatment of various cancers. Most of these agents are delivered by intravenous administration and can produce significant systemic side effects and toxicities that limit their dose and overall use in the treatment of cancer. For example, doxorubicin is a chemotherapy drug indicated for the treatment of breast carcinoma, ovarian carcinoma, thyroid carcinoma, etc. The use of doxorubicin is limited by its irreversible cardiotoxicity, which may be manifested either during, or months to years after termination of therapy. Other side effects commonly associated with chemotherapeutic agents include hematologic toxicity and gastrointestinal toxicity. For example, carmustine is associated with pulmonary, hematologic, gastrointestinal, hepatic, and renal toxicities. The utility of doxorubicin, carmustine, and other chemotherapy agents with a narrow therapeutic index may be improved by delivering the drug at the tumor site in high concentrations using ultrasound-triggered microparticles while reducing the systemic exposure to the drug.

The process for the manufacture of the porous microparticles of the present invention utilizes a different emulsion solvent removal technique from those typically used to produce solid polymer microspheres. Conventionally, the solvent undergoing phase change is evaporated. According to the process of the present invention, solvent removal is effected by sublimation through a lyophilization process. In an evaporation process, mobile polymer molecules in the liquid phase will cohere to form a solid microsphere when solvent is removed. However, the initial freezing step in lyophilization immobilizes the polymer molecules so that when solvent is removed under vacuum, a network of interstitial void spaces surrounded by a web-like polymer structure remains. This porous structure can then be filled with a gas.

The fabrication of the matrix microparticles starts with the preparation of the water-immiscible solvent solution with polymer and drug dissolved therein. Preferred polymers are biodegradable synthetic polymers such as polylactide, polycaprolactone, polyhydroxyvalerate, polyhydroxybutyrate, polyglycolide and copolymers or mixtures of two or more thereof. The requirements for the polymer solvent are that it is substantially water-immiscible and practicably lyophilizable. By practicably lyophilizable it is meant that the solvent freezes at a temperature well above the temperature of a typical lyophilizer minimum condensing capability and that the solvent will sublimate at reasonable rate in vacuo. Suitable solvents include p-xylene, cyclooctane, benzene, decane, undecane, cyclohexane and the like. A preferred polymer is polylactide and the preferred solvent is p-xylene.

A wide variety drugs are suitable for use in the present invention. In a preferred embodiment, the drug is lipophilic and thus relatively soluble in the organic polymer solutions while relatively insoluble in the aqueous phase. In the case of ionic water soluble drugs, the counterion of the drug can greatly impact its lipophilicity. Furthermore, the neutral form of an ionizable molecule is typically more lipophilic than its ionic form. Thus, in another preferred embodiment, a drug that is ionizable in aqueous solution would be incorporated into the microsphere in its neutral, or free base form, or in its ionic form with a counterion that increases the overall lipophilicity of the molecule. As used herein, the word drug refers to chemicals, or biological molecules providing a therapeutic, diagnostic, or prophylactic effect in vivo.

Drugs contemplated for use in the present invention include but are not limited to the following classes: antibiotics, antifungal, anti-inflammatory, antineoplastic, immunosuppressive, antianginal, antiarrhythmic, antiarthritic, antibacterial, anticoagulants, thrombolytic, antifibrinolytic, antiplatelet, antiviral, antimicrobial, anti-infective, steroidal compound, hormones, proteins, and nucleic acids.

The concentration of polymer in solution will dictate the void volume of the end product that will, in turn, impact acoustic performance. A high concentration provides lower void volume and a more acoustically durable microparticle. A lower concentration will result in a more fragile microparticle. Polymer molecular weight also has an effect. A low molecular weight polymer produces a more fragile particle. Optionally, additives may be used in the polymer organic phase. Plasticizers to modify elasticity of the polymer or other agents to affect hydrophobicity of the microparticle can be added to modify the mechanical, and thus acoustic, characteristics of the microparticle. Such plasticizers include the phthalates or ethyl citrates. Agents to modify hydrophobicity include fatty acids and waxes.

The polymer/drug solution is then emulsified in an aqueous phase. The aqueous phase may contain a surface-active component to enhance microdroplet formation and provide emulsion stability for the duration of the fabrication process. Surface-active components include the poloxamers, tweens, and brijs. Also suitable are amphiphilic water-soluble proteins such as gelatin, casein, albumin, or synthetic polymers such as polyvinyl alcohol.

Addition of viscosity enhancers may also be beneficial as an aid in stabilizing the emulsion. Useful viscosity enhancers include carboxymethyl cellulose, dextran, methyl cellulose, hydroxyethyl cellulose, polyvinyl pyrrolidone, and various natural gums such as gum arabic, carrageenan, and guar gum.

The range of ratios of the organic phase to the aqueous phase is typically between 2:1 and 1:20 with a 2:1 to 1:3 ratio range preferred.

If the aqueous phase is also to serve as the suspending medium during the lyophilization step, other components which may be included in the aqueous phase are ingredients suitable as bulking agents such as polyethylene glycol, polyvinyl pyrrolidone, sugars such as glucose, sucrose, lactose, and mannitol. Salts such as sodium phosphate, sodium chloride or potassium chloride may also be included to accommodate tonicity and pH requirements.

A variety of equipment may be used to perform the emulsification step including colloid mills, rotor-stator homogenizers, ultrasonic homogenizers, high pressure homogenizers, microporous membrane homogenizers, with microporous membrane homogenization preferred because the more uniform shearing provides for a more monodisperse population of emulsion droplets.

Size of the droplets formed should be in a range that is consistent with the application. For example, if the microparticles are to be injected intravenously into a subject, then they should have diameters of less than 10 microns in order to pass unimpeded through the capillary network. The size control can be empirically determined by calibration on the emulsification equipment.

If it is desired to provide an optional outer layer of a biologically compatible material, the material is first solubilized in the aqueous phase. This outer layer material will typically be amphiphilic, that is, have both hydrophobic and hydrophilic characteristics. Such materials have surfactant properties and thus tend to be deposited and adhere to interfaces such as the outer surface of the emulsion droplets. Preferred materials are proteins such as collagen, gelatin, casein, serum albumin, or globulins. Human serum albumin is particularly preferred for its blood compatibility. Synthetic polymers may also be used such as polyvinyl alcohol.

The deposited layer of amphiphilic material can be further stabilized by chemical crosslinking. If proteinaceous, suitable chemical crosslinkers include the aldehydes like formaldehyde and glutaraldehyde or the carbodiimides such as dimethylaminopropyl ethylcarbodiimide hydrochloride. To crosslink polyvinyl alcohol, sodium tetraborate may be used.

Provision for the outer layer is preferably achieved by diluting the prepared emulsion into an aqueous bath containing the dissolved chemical crosslinker. This outer crosslinked layer also has the advantage of increasing the stability of the emulsion droplets during the later processing steps.

Provision of a separate outer layer also allows for charge and chemical modification of the surface of the microparticles without being limited by the chemical or physical properties of material present inside the microparticles. Surface charge can be selected, for example, by providing an outer layer of a type “A” gelatin having an isoelectric point above physiological pH or by using a type “B” gelatin having an isoelectric point below physiological pH. The outer surface may also be chemically modified to enhance biocompatibility, such as by pegylation, succinylation, or amidation, as well as being chemically binding to the surface targeting moiety for binding to selected tissues. The targeting moieties may be antibodies, cell receptors, lectins, selectins, integrins, or chemical structures or analogues of the receptor targets of such materials.

Optionally prior to lyophilization, the outer aqueous phase may be replaced by a second aqueous phase. This would allow the first aqueous phase to be optimized for emulsification, while optimizing a second aqueous phase for lyophilization. Replacement may be achieved by means of diafiltration or by centrifugation.

The emulsion is then lyophilized. This involves first freezing both the water immiscible organic phase in the emulsion droplets and the suspending aqueous phase, then removing both phases by sublimation in vacuo. The process produces a dry cake containing porous polymer matrix microparticles with drug incorporated therein.

The drug-containing microparticles are porous and thus can receive a gas. Introducing a selected gas into the lyophilization chamber after the drying step will fill the interstitial voids within the microparticle matrix interior. Alternatively, the gas introduced into the microparticle upon pressurization of the lyophilization chamber can be exchanged for a second gas.

Any gas may be used, but biologically inert gases such as air, nitrogen, helium, oxygen, xenon, argon, helium, carbon dioxide, and halogenated hydrocarbons such as perfluorobutane, perfluoropropane or sulfur halides such as sulfur hexafluoride are preferred. Depending upon the application, one may select the gas based on its solubility in blood. For example, perfluorocarbons have low solubility while carbon dioxide has very high solubility. Such differences in solubility will influence the acoustic performance of the microparticle.

During the lyophilization process the polymer solvent and the water of the excipient suspending medium are removed at reduced pressure by sublimation to form a population of substantially solvent free microparticles having a polymer matrix interior. The incorporated drug will remain within the polymer matrix until the microparticle is made to rupture in the bloodstream using ultrasound.

In clinical use, the dry lyophilized product is reconstituted by addition of an aqueous solution and the resulting microparticle suspension intravenously injected. As the drug-containing gas-filled microparticles circulate systemically, their presence at the site of delivery can be monitored using an ultrasound device operating at power levels below what is required to rupture the microparticles. Then at the appropriate time, when a required concentration of microparticles is present at the site, the power level can be increased to a level sufficient to rupture and flood the microparticles, thus triggering the release of the drug payload.

Preferably, the rupture of the drug-containing microparticles is achieved using ultrasound scanning devices and employing transducers commonly utilized in diagnostic contrast imaging. In such instances a single ultrasound transducer may be employed for both imaging and rupturing of the microparticles by focusing the beam upon the target site and alternately operating at low and high power levels as required by the application.

Alternatively, a plurality of transducers focused at the region may be used so that the additive wave superposition at the point of convergence creates a local intensity sufficient to flood the microparticles. A separate imaging transducer may be used to image the region for treatment.

While not required, it is preferred that the microparticles be rupturable for drug release at power levels below the clinically accepted levels for diagnostic imaging. Specific matching of ultrasound conditions and microparticle response to such conditions achieve controlled release conditions. Preferred acoustic conditions for rupture are those at a power, frequency, and waveform sufficient to provide a mechanical index from about 0.1 to about 1.9.

The following examples are provided by way of illustration and are not intended to limit the invention in any way.

### EXAMPLE 1

### Fabrication of Gas-Filled Microparticles Having a Polymer Matrix Interior

An aqueous solution of 1% polyvinyl alcohol and 2.8% mannitol was prepared. Separately, a polymer solution containing 6% poly DL-lactide in p-xylene was prepared. To 40.0 g of the polymer solution was combined 50.0 g of the aqueous solution in a jacketed beaker maintained at 30° C. The mixture was then emulsified to create an oil-in-water emulsion using a circulating system consisting of a peristaltic pump and a sintered metal filter having a nominal pore size of 7 microns. After circulating for 6 minutes, 35.0 g of the resultant emulsion was diluted with 177.9 g of a 2.8% mannitol solution also maintained at 30° C. After 15 minutes of continuous stirring, a portion of the diluted emulsion was dispensed into 10 ml vials and then lyophilized. After the drying cycle was completed, nitrogen gas was introduced into the lyophilization chamber to a pressure slightly below atmospheric and the vials stoppered.

Microscopic inspection of the reconstituted product revealed discrete gas-filled microparticles.

### EXAMPLE 2

### Fabrication of Gas-Filled Microparticles Having a Polymer Matrix Interior

A solution of 5.4% human serum albumin (hsa) was prepared by dilution of a 25% solution and the pH adjusted to 4 with HCl. Separately, 0.99 gm poly D-L lactide was dissolved in 29.0 gm p-xylene. In a jacketed beaker maintained at 40° C., the polylactide solution was combined with 30 gms of the previously prepared hsa solution and a coarse emulsion was formed using magnetic stirring. A peristaltic pump was used to pump the coarse emulsion through a porous sintered metal filter element with a 2 μm nominal pore size. The emulsion was recirculated through the element for approximately 15 minutes until the average droplet size was less than 10 microns. The emulsion was diluted into 350 ml of a 40° C. aqueous bath containing 1.0 ml of a 25% glutaraldehyde solution and 1.4 ml of 1N NaOH. After 15 minutes, 0.75 gm of poloxamer 188 surfactant was dissolved into the aqueous bath. The emulsion microdroplets were retrieved by centrifugation at 2000 rpm for 10 minutes, formulated into an aqueous solution containing polyethylene glycol, glycine, and poloxamer 188, dispensed into 10 ml vials, and then lyophilized. After the drying cycle was completed, nitrogen gas was introduced into the lyophilization chamber to a pressure slightly less than atmospheric and the vials were stoppered.

Microscopic inspection of the reconstituted product revealed discrete gas-filled microparticles.

### EXAMPLE 3

### Fabrication of Gas-Filled Microparticles Having a Polymer Matrix Interior and Containing a Dye

A solution of 1% Fluorescent Yellow Dye R (Keystone PIN 806-043-50) and 6% poly DL-lactide was prepared in xylene. Separately, an aqueous solution containing 1% polyvinyl alcohol and 2.8% mannitol was prepared. In a jacketed beaker maintained at 30° C., 40 gm of the dye containing polylactide solution was combined with 50 gms of the prepared aqueous solution and a coarse emulsion was formed using magnetic stirring. A peristaltic pump was used to pump the coarse emulsion through a porous sintered metal filter element with a 7 μm nominal pore size. The emulsion was recirculated through the element for approximately 6 minutes until the average droplet size was less than 10 microns. The emulsion was diluted with stirring into 400 ml of a 30° C. aqueous bath containing 2.8% mannitol. After 15 minutes, the emulsion was dispensed into 10 mL vials and lyophilized. After the drying cycle was completed, nitrogen gas was introduced into the lyophilization chamber to a pressure slightly less than atmospheric and the vials were stoppered.

Microscopic inspection of the reconstituted product revealed discrete gas-filled microparticles.

### EXAMPLE 4

### Release of Dye Using Ultrasound

Four vials of product manufactured in accordance with Example 3 were each reconstituted with 2 mL of a 0.25% Poloxamer 188 and 1% isopropyl alcohol solution (wash solution). Two of the vials were pooled and used as the control sample. The remaining two vials were combined and used as the experimental sample. Each sample was further diluted with 3 mL wash solution and placed in a test tube. All samples were centrifuged at 1500 rpm for 25 minutes. The cream layer containing the matrix microparticles were retrieved and resuspended with vortex mixing in 3 mL of wash solution. The washing procedure was repeated two more times. The final washed cream was then suspended in a total volume of 6.5 mL of wash solution.

The control sample remained undisturbed for 2 hours to allow the dye-containing microparticles to float. The subnatant was removed and centrifuged at 14,000 rpm for 2 minutes. The experimental sample was sonicated for 1 minute on level 5 using a Virtis Virsonic hand-held sonicator. Microscopic inspection of the suspension revealed that the microparticles had become flooded as a result of the sonication procedure. The suspension was centrifuged at 14,000 rpm for 2 minutes. The supernatants from both the control and the experimental samples were read on a spectrophotometer using a wavelength of 463 nm. A standard curve of Fluorescent Yellow Dye R concentration verses absorbance at 463 nm was generated and was found to be linear. The amount of dye in each sample was calculated, from the absorbance using the standard curve. The concentration of Fluorescent Yellow Dye R in the control was 1.76 μg/mL while the experimental sample contained 5.06 μg/mL thus demonstrating the release of dye from the prepared polymer matrix microparticles using ultrasound.

### EXAMPLE 5

### Fabrication of Gas-Filled Microparticles Having a Polymer Matrix Interior and Containing Oxybutynin

A solution of 1% oxybutynin and 6% poly DL-lactide was prepared in xylene. Separately, an aqueous solution containing 1% polyvinyl alcohol and 2.8% mannitol was prepared and the pH was adjusted to 8 using NaOH. In a jacketed beaker maintained at 30° C., 40 gm of the oxybutynin containing polylactide solution was combined with 50 gms of the prepared aqueous solution and a coarse emulsion was formed using magnetic stirring. A peristaltic pump was used to pump the coarse emulsion through a porous sintered metal filter element with a 7 μm nominal pore size. The emulsion was recirculated through the element for approximately 6 minutes until the average droplet size was less than 10 microns. The emulsion was diluted with stirring into 400 ml of a 30° C. aqueous bath containing 2.8% mannitol and at a pH of 8. After 15 minutes of stirring, the emulsion was dispensed into 10 mL vials and lyophilized. After the drying cycle was completed, nitrogen gas was introduced into the lyophilization chamber to a pressure slightly less than atmospheric and the vials were stoppered.

Microscopic inspection of the reconstituted product revealed discrete gas-filled microparticles.

### EXAMPLE 6

### Release of Oxybutynin Using Ultrasound

Four vials containing product manufactured in accordance with Example 5 were each reconstituted with 10 mL of 20 mM KPO₄ at pH 3.5 (wash solution). The oxybutynin containing microparticles from two of the vials were retrieved by centrifugation at 1500 rpm for 15 minutes. The microparticles were resuspended in 10 mL of wash solution and vortexed. The washing procedure was repeated two more times. The final washed matrix microparticles were resuspended in a total volume of 10 mL of wash solution. The remaining two vials were not washed. One vial from each set was designated the control sample and the other vial was the experimental sample.

The controls remained undisturbed for 80 minutes to allow the matrix microparticles to float. The subnatant was removed and centrifuged at 14,000 rpm for 2 minutes. The experimental samples were each sonicated for 1 minute on level 5 using a Virtis Virsonic hand-held sonicator. Microscopic inspection of the suspensions revealed that the microparticles had become flooded as a result of the sonication procedure. The suspensions were centrifuged at 14,000 rpm for 2 minutes. All four of the resulting supernatants were retrieved and analyzed by reverse phase HPLC. A standard curve of area verses oxybutynin concentration was generated and was found to be linear. The amount of oxybutynin in each sample was calculated from the area using the standard curve.

For the washed samples, the control sample contained 0.03 mg oxybutynin/vial, while the experimental sample contained 1.05 mg/vial. For the unwashed samples, the control sample had 0.65 mg oxybutynin/vial and the experimental sample had 1.93 mg/vial. Theoretical loading was 2.4 mg/vial.

When compared to theoretical loading, 80% of the total oxybutynin was recovered after sonication. Of the recovered amount, the oxybutynin released due to sonication was 66%, while the amount of burst release was only 34%. After the burst release was removed by the washing, the control contained almost no oxybutynin thus showing little to no leakage of drug after reconstitution. The washing only eliminated the burst release and did not affect any of the encapsulated oxybutynin.

### EXAMPLE 7

### Contrast Efficacy of Gas-Filled Microparticles Having a Polymer Matrix Interior

An Agilent 5500 ultrasonic scanner was used for this study to measure the acoustic backscatter and fragility from a suspended matrix particle. This scanner has the capability of measuring the acoustic density (AD) as a function of time within a region of interest (ROI) displayed on the video monitor. The scanner was set to the 2D harmonic mode with send frequency of 1.8 MHz and receive frequency of 3.6 MHz. The test cell was a 2 cm diameter tube running the length of a Doppler flow phantom manufactured by ATL Laboratories of Bridgeport, Conn. Microparticle agent made in accordance with Example 1 was first reconstituted with deionized water. The resulting suspension was diluted into a 1 liter beaker containing water and then circulated through the flow phantom using a peristaltic pump (Masterflex L/S manufactured by Cole-Parmer). To insure that the agent remained uniformly suspended in the beaker, mixing using a VWR Dylastir magnetic stirrer in conjunction with a 2 cm coated plastic stir bar was maintained throughout the duration of the testing. When data was to be collected, the pump was turned off resulting in no flow within the phantom.

The scanner transducer (s4 probe) was placed directly over the flow phantom within a water-well designed into the phantom. It was oriented 90 degrees to the flow axis such that the image of the flow tube on the monitor was circular. The ROI (21×21) was positioned by the operator within the image of the tubing lumen to be at the top center about 1 mm away from the top wall and free of any bright echoes caused by the wall. The scanner was set to the acoustic densitometry (AD) mode. This mode permits the scanner to read the mean densitometry within the ROI as a function of time using a triggered mode. The triggering interval was selected to be 200 milliseconds. For each test run, suspended agent was circulated into the flow tube and then flow was discontinued. Using a triggering interval of 200 milliseconds, the sample was then insonated and the acoustic densitometry within the ROI was measured at each frame. The tests were repeated at several scanner power levels.

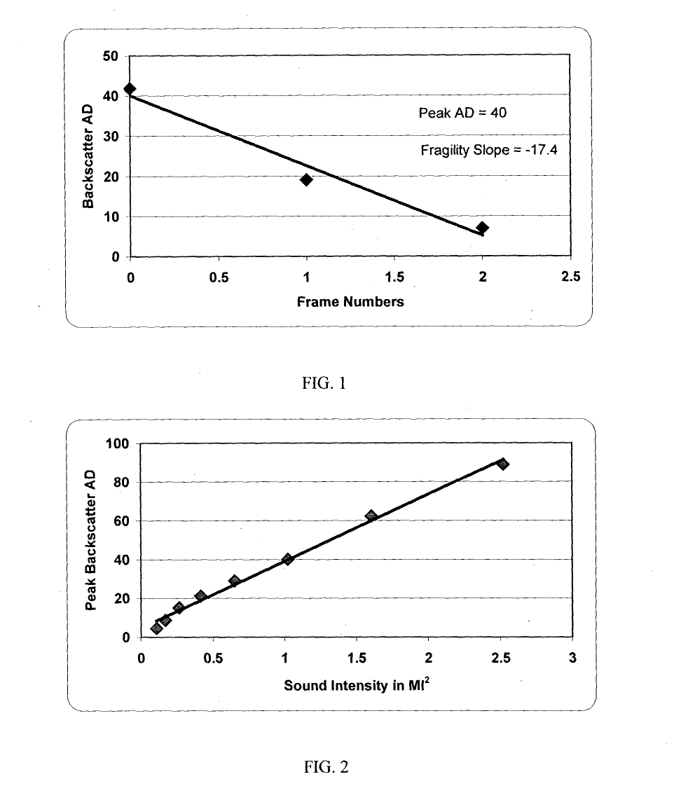

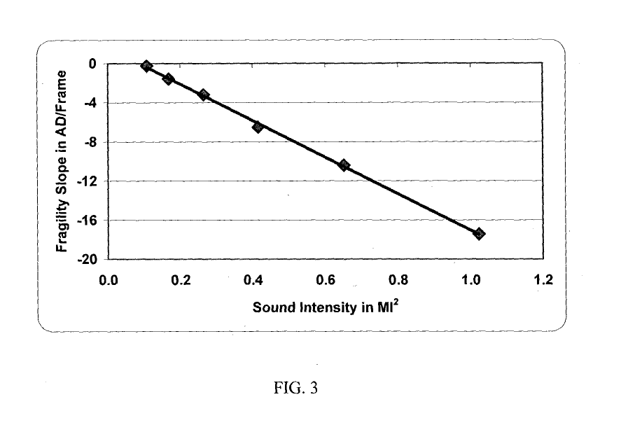

From the AD decay curve at each power setting, a linear regression curve was fit through the first three or four data points. The zero time intercept provides the peak backscatter produced by the agent as seen in FIG. 1. The slope of that curve is identified as the fragility slope and is a measure of the fragility of the agent. These two measurements are plotted respectively against intensity in FIGS. 2 and 3. Note that the backscatter of the matrix microparticle increases linearly with ultrasound intensity (FIG. 2) and this is considered typical behavior. The plot of fragility slope (FIG. 3) provides some additional information regarding the agent. First, it indicates that the agent is rupturing upon exposure to ultrasound. Secondly, its intercept with the x-axis in FIG. 3 identifies the point where it begins to rupture. Thus for this agent, at fragility slope −17.4 the agent begins to fail at an MI² value of intensity of 0.0868, which is a value of MI of 0.295. Thus for values of MI less than 0.295, the agent will not fail and therefore if drug were encapsulated within it would not be released.

What is claimed is:

1. A method of preparing drug-containing gas-filled polymer matrix microparticles useful for delivering a drug to an organ or tissue using ultrasound comprising the steps of:
a. dissolving a polymer and a drug in a substantially water-immiscible solvent to form a polymer solution;
b. emulsifying said polymer solution in an aqueous medium to form an oil-in-water emulsion comprising an aqueous phase and nonaqueous phase droplets;
c. reducing the temperature of said oil-in-water emulsion sufficiently to freeze said aqueous phase and nonaqueous phase droplets;
d. removing the water from said aqueous phase and said solvent from said nonaqueous phase droplets by sublimation to form drug-containing porous polymer matrix microparticles;
e. introducing a gas into said microparticles.

2. A method according to claim 1 wherein said aqueous medium contains a biologically compatible amphiphilic material and further comprising the step subsequent to step b of diluting said emulsion into a second aqueous medium containing a chemical crosslinking agent thereby forming an outer layer of crosslinked biologically compatible amphiphilic material around said droplets.

3. A method according to claim 1 further comprising the step subsequent to step b of exchanging or partially exchanging said aqueous phase by a second aqueous medium.

4. A method according to claim 1 wherein said polymer comprises a biodegradable synthetic polymer.

5. A method according to claim 4 wherein said polymer is selected from the group consisting of polylactide, polycaprolactone, polyglycolide, polyhydroxybutyrate, polyhydroxyvalerate, and copolymers or mixtures of any two or more thereof.

6. A method according to claim 5 wherein said polymer comprises polylactide.

7. A method according to claim 2 wherein said biologically compatible amphiphilic material comprises a protein.

8. A method according to claim 7 wherein said biologically compatible amphiphilic material is selected from the group consisting of serum albumin, gelatin, collagen, globulins, casein, and combinations of two or more thereof.

9. A method according to claim 8 wherein said biologically compatible amphiphilic material comprises serum albumin.

10. A method according to claim 2 wherein said crosslinking agent comprises glutaraldehyde.

11. A method according to claim 1 wherein said water-immiscible solvent is selected from the group consisting of xylene, benzene, cyclohexane, cyclooctane, and combinations of two or more thereof.

12. A method according to claim 11 wherein said organic solvent comprises xylene.

13. A method according to claim 1 wherein said gas is selected from the group consisting of air, nitrogen, oxygen, argon, helium, carbon dioxide, xenon, a sulfur halide, and a halogenated hydrocarbon.

14. A method according to claim 13 wherein said gas comprises nitrogen.

15. A method according to claim 1 wherein said drug comprises an antibiotic, antifungal, anti-inflammatory, antineoplastic, immunosuppressive, antianginal, antiarrhythmic, antiarthritic, antibacterial, anticoagulant, thrombolytic, antifibrolytic, antiplatelet, antiviral, antimicrobial, anti-infective, steroidal, hormonal, proteinaceous or nucleic acid drugs.

16. A method according to claim 15 wherein said drug is lipophilic.

17. A method according to claim 15 wherein said drug is ionizable in aqueous media.

18. A method for delivery of a drug to an organ or tissue using ultrasound comprising the steps of:
a. introducing a microparticle composition according to claim 1 into said organ or tissue,
b. applying an ultrasound signal to said organ or tissue at a power intensity sufficient to induce rupture of said microparticles,
a. maintaining said power intensity until at least a substantial number of the microparticles are ruptured.

19. A method according to claim 18 comprising, after step a) the step of the location of said microparticles within said organ or tissue by applying an ultrasound signal to said region of interest at a power intensity below that which is sufficient to rupture said microparticles.

20. A method according to claim 18 wherein said ultrasound power intensity sufficient to induce rupture of said microparticles is at a mechanical index between about 0.1 and about 1.9.

21. A composition for in vivo drug delivery comprising gas-filled porous polymer matrix microparticles having an outer surface of biologically compatible amphiphilic material, a polymer matrix interior containing gas and a drug.

22. A composition according to claim 21 wherein said polymer comprises a biodegradable synthetic polymer.

23. A composition according to claim 22 wherein said polymer is selected from the group consisting of polylactide, polycaprolactone, polyglycolide, polyhydroxybutyrate, polyhydroxyvalerate, and copolymers or mixtures of any two or more thereof.

24. A composition according to claim 23 wherein said polymer comprises polylactide.

25. A composition according to claim 21 wherein said biologically compatible amphiphilic material comprises a protein.

26. A composition according to claim 25 wherein said biologically compatible amphiphilic material is selected from the group consisting of serum albumin, gelatin, collagen, globulins, casein, and combinations of two or more thereof.

27. A composition according to claim 26 wherein said biologically compatible amphiphilic material comprises serum albumin.

28. A composition according to claim 21 wherein said amphiphilic material is crosslinked with glutaraldehyde.

29. A composition according to claim 21 wherein said gas is selected from the group consisting of air, nitrogen, oxygen, argon, helium, carbon dioxide, xenon, a sulfur halide, and a halogenated hydrocarbon.

30. A composition according to claim 29 wherein said gas comprises nitrogen.

31. A composition according to claim 21 wherein said drug comprises an antibiotic, antifungal, anti-inflammatory, antineoplastic, immunosuppressive, antianginal, antiarrhythmic, antiarthritic, antibacterial, anticoagulant, thrombolytic, antifibrolytic, antiplatelet, antiviral, antimicrobial, anti-infective, steroidal, hormonal, proteinaceous or nucleic acid drugs.

32. A composition according to claim 31 wherein said drug is lipophilic.

33. A composition according to claim 31 wherein said drug is ionizable in aqueous media.
